(19) United States
(12) Patent Application Publication
(10) Pub. No.: US 2023/0189980 A1
(43) Pub. Date: Jun. 22, 2023

(54) NASAL CAVITY CLEANER AND ORAL CAVITY CLEANER

(71) Applicants: Tomotaka Iwamoto, Kanagawa (JP); Shinya Sonohara, Nagano (JP)

(72) Inventor: Tomotaka Iwamoto, Kanagawa (JP)

(21) Appl. No.: 18/083,499

(22) Filed: Dec. 17, 2022

(30) Foreign Application Priority Data
Dec. 17, 2021 (JP) 2021204958
Dec. 17, 2021 (JP) 2022078693
Dec. 17, 2021 (JP) 2022158632

(51) Int. Cl. A46B 15/00; A47K 7/02

(52) U.S. Cl. CPC A46B 15/0055; A47K 7/02

(57) ABSTRACT

A nasal cavity cleaner is an instrument to be inserted into the nasal cavity for cleaning the nasal cavity. The nasal cavity cleaner includes a grip section to be gripped by a user, a shaft-shaped brush holding section extending from the grip section, and a plurality of brush pieces stacked on each other and held by the brush holding section. Each of the plurality of brush pieces has an annular section and a plurality of brush bristles provided on an outer peripheral surface of the annular section. Further, each of the plurality of brush pieces is held by the brush holding section inserted through the annular section. The brush holding section has a locking section that is locked to the inner peripheral surface of the annular section of the brush piece held by the brush section, thereby suppressing a rotation of the brush piece.

### CROSS REFERENCE TO RELATED APPLICATION

This application takes priority from and claims the benefit of Japanese Patent Application No. Japanese Patent Application No. 2021-204958 filed on Dec. 17, 2021, Japanese Patent Application No. 2022-078693 filed on Dec. 17, 2021 (Divisional application of No. 2021-204958), Japanese Patent Application No. 2022-158632 filed on Dec. 17, 2021 (Divisional application of No. 2022-078693), the contents of which are herein incorporated by reference.

### BACKGROUND OF THE INVENTION

### Field of the Invention

The present disclosure relates to a nasal cavity cleaner and an oral cavity cleaner.

### Description of the Related Art

Foreign substances such as secretions from a nasal cavity or foreign substances having entered from the outside may accumulate in the nasal cavity. These foreign substances are desired to be removed. For example, when nasal mucus is secreted from the nasal cavity, the removal of the nasal mucus relieves discomfort. Further, according to the results of intensive research made by the inventor of the present disclosure, when an allergen, or an allergenic substance, that can cause an allergic reaction invades the nasal cavity enters the nasal cavity, sufficient removal of the allergen can be expected to alleviate the allergic reaction.

As an instrument for removing foreign substances in the nasal cavity, a nasal cavity cleaner as disclosed in Patent Document 1 is known to public, for example. This nasal cavity cleaner is a so-called 360-degree brush with a plurality of brush pieces including a plurality of radially arranged brush bristles being stacked on each other and held. Each of the plurality of brush pieces has a circular opening in the center, is fitted into a cylindrical shaft-shaped brush holding section formed to have approximately the same diameter as the opening, and accordingly constitutes a brush section to be inserted into the nasal cavity. Moreover, an oral cavity cleaner for cleaning the oral cavity is also known as an instrument similar to this nasal cavity cleaner.

The contents of Utility Model Registration No. 3232109 are incorporated herein by reference in their entirety.

### SUMMARY OF THE INVENTION

### Problems to be Solved by the Invention

When the nasal cavity cleaner as described above is rotated around the central axis line of the brush holding section while the brush section is inserted into the nasal cavity, it removes foreign substances in the nasal cavity with the brush bristles. However, when this nasal cavity cleaner is rotated around the central axis line inside the nasal cavity, the brush holding section may fail to hold the brush pieces due to a friction between the inner wall of the nasal cavity and the brush bristles, and accordingly may rotate idly in relation to the brush pieces. As a result, even if a grip section and the brush holding section are rotated, the brush section may remain stationary in the nasal cavity failing to rotate, which may make it impossible to effectively clean the nasal cavity. Similarly, the brush section of the oral cavity cleaner may remain stationary failing to rotate, which may make it impossible to effectively clean the oral cavity.

Therefore, the nasal cavity cleaner and the oral cavity cleaner according to the present disclosure aim to suppress the idle rotation of the brush holding section in relation to the brush pieces.

### Means of Solving the Problems

A nasal cavity cleaner according to one embodiment of the present disclosure is a nasal cavity cleaner so as to be inserted into a nasal cavity for cleaning the nasal cavity. The nasal cavity cleaner includes a grip section to be gripped by a user and a shaft-shaped brush section extending from the grip section and a plurality of brush pieces stacked on each other and held by the brush holding section. Each of the plurality of brush pieces includes an annular section formed in an annular shape and a plurality of brush bristles provided on an outer peripheral surface of the annular section. Each of the plurality of brush pieces is held by the brush holding section inserted through the annular section. The brush holding section includes a locking section locked to an inner peripheral surface of the annular section of the brush piece held by the brush holding section to suppress a rotation of the brush piece around the central axis line.

According to this nasal cavity cleaner, the shaft-shaped brush holding section is inserted through the annular section of the plurality of brush pieces stacked on each other, thereby configuring the brush section to be inserted into the nasal cavity. Here, the brush holding section is provided with the locking section locked to the annular section of the brush piece. Therefore, when the nasal cavity cleaner is rotated around the central axis line in the nasal cavity, a friction between the inner wall of the nasal cavity and the brush bristles can suppress the failure of the brush holding section to hold the brush pieces, enabling the brush pieces to rotate integrally with the brush holding section. This enables the suppression of idle rotation of the brush holding section in relation to the brush pieces.

In the nasal cavity cleaner according to one embodiment of the present disclosure, the plurality of brush bristles may be provided radially outward from the outer peripheral surface of the annular section over the entire circumference of the outer peripheral surface of the annular section. This enables effective removal of foreign substances in the nasal cavity by rotating the nasal cavity cleaner around the central axis line while the brush section is inserted into the nasal cavity.

In the nasal cavity cleaner according to one embodiment of the present disclosure, the annular section may be formed in an annular shape. The brush holding section may include a cylindrical base section and a rib-shaped locking section extending on the outer peripheral surface of the base section along the central axis line of the brush holding section and having the outer diameter from the central axis line of the brush holding section to the top of the locking section larger than the inner diameter of the annular section. The brush holding section may be inserted through the annular section by press fitting, thereby locking the locking section to the inner peripheral surface of the annular section. According to this, the brush piece can be locked to the brush holding section more firmly, so that the idle rotation of the brush holding section in relation to the brush piece can be suppressed more reliably.

In the nasal cavity cleaner according to one embodiment of the present disclosure, on a cross-section perpendicular to the central axis line of the brush holding section, the top of the locking section may be positioned between two interfaces between the locking section and the base section when viewed in at least one direction perpendicular to the central axis line. This facilitates the removal of the brush holding section from the mold when manufacturing the brush holding section, for example, by injection molding.

In the nasal cavity cleaner according to one embodiment of the present disclosure, the inner peripheral surface of the annular section of each of the plurality of brush pieces may be pressed and expanded by the locking section while the brush holding section is inserted through the annular section. This facilitates the manufacturing of the brush piece as there is no need of forming in advance a recess corresponding to the locking section on the inner peripheral surface of the annular section. In addition, a pressing force generated between the locking section and the inner peripheral surface of the annular section can lock the brush piece more firmly to the brush holding section, thereby enabling more reliable suppression of idle rotation of the brush holding section in relation to the brush piece.

In the nasal cavity cleaner according to one embodiment of the present disclosure, the locking section may be crushed by the inner peripheral surface of the annular section while the brush holding section is inserted through the annular portion. This facilitates the manufacturing of the brush piece as there is no need of forming in advance a recess corresponding to the locking section on the inner peripheral surface of the annular section. In addition, a pressing force generated between the locking section and the inner peripheral surface of the annular section can lock the brush piece more firmly to the brush holding section, thereby enabling more reliable suppression of idle rotation of the brush holding section in relation to the brush piece.

In the nasal cavity cleaner according to one embodiment of the present disclosure, the annular section may be formed in a polygonal annular shape so that at least the inner peripheral surface has a polygonal shape. The brush holding section may be formed in a polygonal columnar shape fitting into the annular section. The locking section may be corners of a cross-section perpendicular to the central axis line of the brush holding section. According to this, the shapes of the locking section and the inner peripheral surface of the annular section match each other, thereby enabling more reliable suppression of idle rotation of the brush holding section in relation to the brush pieces.

In the nasal cavity cleaner according to one embodiment of the present disclosure, the brush holding section may be loosely fitted into the annular section. This facilitates insertion of the brush holding section into the annular section.

In the nasal cavity cleaner according to one embodiment of the present disclosure, each of the plurality of brush pieces stacked on each other may be in contact with another adjacent one of the brush pieces on the side surfaces of the annular sections of the brush pieces adjacent to each other. According to this, a frictional force generated between the side surfaces of the annular sections of the brush pieces adjacent to each other suppresses the relative rotation of the brush pieces around the central axis line. As a result, the plurality of brush pieces are cooperatively locked to the brush holding section, thereby enabling more reliable suppression of idle rotation of the brush holding section in relation to the brush pieces.

In the nasal cavity cleaner according to one embodiment of the present disclosure, each of the plurality of brush pieces may include a convex section formed on the side surface of the annular section. According to this, the side surfaces of the annular sections of the brush pieces adjacent to each other are firmly connected to each other by the convex section to suppress the relative rotation of the brush pieces around the central axis line. As a result, the plurality of brush pieces are cooperatively locked to the brush holding section, thereby enabling more reliable suppression of idle rotation of the brush holding section in relation to the brush pieces.

In the nasal cavity cleaner according to one embodiment of the present disclosure, the brush holding section includes a wall section on the grip section side on which the annular section of the brush piece abuts when the brush holding section is first inserted through the annular section, and a pressurizing lid section for pressurizing and holding the annular section of the brush piece along the central axis line toward the grip section when the brush holding section is last inserted through the annular section. According to this, the plurality of brush pieces are pressurized and held by the wall section and the pressurizing lid section of the brush holding section, thereby enabling more reliable suppression of idle rotation of the brush holding section in relation to the brush pieces.

In the nasal cavity cleaner according to one embodiment of the present disclosure, the pressurizing lid section may be formed by ultrasonic welding. This facilitates the manufacturing of the nasal cavity cleaner having the above-described configuration.

In the nasal cavity cleaner according to one embodiment of the present disclosure, at least at the end opposite to the grip section, the plurality of brush bristles may be formed to include the brush bristles shorter in length from the grip section side toward the side opposite to the grip section. According to this, the brush section is in a tapered shape, thereby facilitating the insertion of the brush section into the nasal cavity. Furthermore, when the brush piece positioned at the end opposite to the grip section is supported by a small-diameter lid section, it is hard to ensure the frictional force between the side surface of the annular section of the brush piece and the lid section. In this case, shortening of the brush bristles of the brush piece in length reduces a torque acting on the brush piece owing to the friction between the inner wall of the nasal cavity and the brush bristles, thereby enabling the suppression of the excess of this torque over the frictional force between the side surface of the annular section and the lid section. This enables more reliable suppression of the idle rotation of the brush holding section in relation to the brush pieces.

An oral cavity cleaner according to one embodiment of the present disclosure is an oral cavity cleaner so as to be inserted into an oral cavity for cleaning the oral cavity. The oral cavity cleaner includes a grip section to be gripped by a user and a shaft-shaped brush section extending from the grip section and a plurality of brush pieces stacked on each other and held by the brush holding section. Each of the plurality of brush pieces includes an annular section formed in an annular shape and a plurality of brush bristles provided on an outer peripheral surface of the annular section. Each of the plurality of brush pieces is held by the brush holding section inserted through the annular section. The brush holding section has an locking section locked to an inner peripheral surface of the annular section of the brush piece held by the brush holding section to suppress a rotation of the brush piece around the central axis line.

According to this oral cavity cleaner, the shaft-shaped brush holding section is inserted through the annular sections of the plurality of brush pieces stacked on each other, thereby configuring the brush section to be inserted into the oral cavity. Here, the brush holding section is provided with the locking section locked to the annular section of the brush piece. Therefore, when the oral cavity cleaner is rotated around the central axis line in the oral cavity, the friction between the inner wall of the oral cavity and the brush bristles can suppress the failure of the brush holding section to hold the brush pieces, enabling the brush pieces to rotate integrally with the brush holding section. This enables the suppression of idle rotation of the brush holding section in relation to the brush pieces.

In the oral cavity cleaner according to one embodiment of the present disclosure, the plurality of brush bristles may be provided radially outward from the outer peripheral surface of the annular section over the entire circumference of the outer peripheral surface of the annular section. This enables effective removal of foreign substances in the oral cavity by rotating the oral cavity cleaner around the central axis line while the brush section is inserted into the oral cavity.

In the oral cavity cleaner according to one embodiment of the present disclosure, the annular section may be formed in an annular shape. The brush holding section may include a cylindrical base section and a rib-shaped locking section extending on the outer peripheral surface of the base section along the central axis line of the brush holding section and having the outer diameter from the central axis line of the brush holding section to the top of the locking section larger than the inner diameter of the annular section. The brush holding section may be inserted through the annular section by press fitting, thereby locking the locking section to the inner peripheral surface of the annular section. According to this, the brush piece can be locked to the brush holding section more firmly, so that the idle rotation of the brush holding section in relation to the brush piece can be suppressed more reliably.

In the oral cavity cleaner according to one embodiment of the present disclosure, on a cross-section perpendicular to the central axis line of the brush holding section, the top of the locking section may be positioned between two interfaces between the locking section and the base section when viewed in at least one direction perpendicular to the central axis line. This facilitates the removal of the brush holding section from the mold when manufacturing the brush holding section, for example, by injection molding.

In the oral cavity cleaner according to one embodiment of the present disclosure, the inner peripheral surface of the annular section of each of the plurality of brush pieces may be pressed and expanded by the locking section while the brush holding section is inserted through the annular section. This facilitates the manufacturing of the brush piece as there is no need of forming in advance a recess corresponding to the locking section on the inner peripheral surface of the annular section. In addition, a pressing force generated between the locking section and the inner peripheral surface of the annular section can lock the brush piece more firmly to the brush holding section, thereby enabling more reliable suppression of idle rotation of the brush holding section in relation to the brush piece.

In the oral cavity cleaner according to one embodiment of the present disclosure, the locking section may be crushed by the inner peripheral surface of the annular section while the brush holding section is inserted through the annular portion. This facilitates the manufacturing of the brush piece as there is no need of forming in advance a recess corresponding to the locking section on the inner peripheral surface of the annular section. In addition, a pressing force generated between the locking section and the inner peripheral surface of the annular section can lock the brush piece more firmly to the brush holding section, thereby enabling more reliable suppression of idle rotation of the brush holding section in relation to the brush piece.

In the oral cavity cleaner according to one embodiment of the present disclosure, the annular section may be formed in a polygonal annular shape so that at least the inner peripheral surface has a polygonal shape. The brush holding section may be formed in a polygonal columnar shape fitting into the annular section. The locking section may be corners of a cross-section perpendicular to the central axis line of the brush holding section. According to this, the shapes of the locking section and the inner peripheral surface of the annular section match each other, thereby enabling more reliable suppression of idle rotation of the brush holding section in relation to the brush pieces.

In the oral cavity cleaner according to one embodiment of the present disclosure, the brush holding section may be loosely fitted into the annular section. This facilitates insertion of the brush holding section into the annular portion.

In the oral cavity cleaner according to one embodiment of the present disclosure, each of the plurality of brush pieces stacked on each other may be in contact with another adjacent one of the brush pieces on the side surfaces of the annular sections of the brush pieces adjacent to each other. According to this, a frictional force generated between the side surfaces of the annular sections of the brush pieces adjacent to each other suppresses the relative rotation of the brush pieces adjacent to each other around the central axis line. As a result, the plurality of brush pieces are cooperatively locked to the brush holding section, thereby enabling more reliable suppression of idle rotation of the brush holding section in relation to the brush pieces.

In the oral cavity cleaner according to one embodiment of the present disclosure, each of the plurality of brush pieces may include a convex section formed on the side surface of the annular section. According to this, the side surfaces of the annular sections of the brush pieces adjacent to each other are firmly connected to each other by the convex section to suppress the relative rotation of the brush pieces around the central axis line. As a result, the plurality of brush pieces are cooperatively locked to the brush holding section, thereby enabling more reliable suppression of idle rotation of the brush holding section in relation to the brush pieces.

In the oral cavity cleaner according to one embodiment of the present disclosure, the brush holding section includes a wall section on the grip section side on which the annular section of the brush piece abuts when the brush holding section is first inserted through the annular section, and a pressurizing lid section for pressurizing and then holding the annular section of the brush piece along the central axis line toward the grip section when the brush holding section is last inserted through the annular section. According to this, the plurality of brush pieces are pressurized and then held by the wall section and the pressurizing lid section of the brush holding section, thereby enabling more reliable suppression of idle rotation of the brush holding section in relation to the brush pieces.

In the oral cavity cleaner according to one embodiment of the present disclosure, the pressurizing lid section may be formed by ultrasonic welding. This facilitates the manufacturing of the oral cavity cleaner having the above-described configuration.

In the oral cavity cleaner according to one embodiment of the present disclosure, at least at the end opposite to the grip section, the plurality of brush pieces may be formed to include the brush bristles shorter in length from the grip section side toward the side opposite to the grip section. According to this, the brush section is in a tapered shape, thereby facilitating the insertion of the brush section into the oral cavity. Furthermore, when the brush piece positioned at the end opposite to the grip section is supported by a small-diameter lid section, it is hard to ensure the frictional force between the side surface of the annular section of the brush piece and the lid section. In this case, shortening of the brush bristles of the brush piece in length reduces a torque acting on the brush piece owing to the friction between the inner wall of the nasal cavity and the brush bristles, thereby enabling the suppression of the excess of this torque over the frictional force between the side surface of the annular section and the lid section. This enables more reliable suppression of the idle rotation of the brush holding section in relation to the brush pieces.

### Effects of the Invention

In this manner, the nasal cavity cleaner and the oral cavity cleaner according to the present disclosure can suppress the idle rotation of the brush holding section in relation to the brush pieces.

### BRIEF DESCRIPTION OF THE DRAWINGS

FIG. 1 is a view of a nasal cavity cleaner according to a first embodiment;

FIG. 2 is a view of a grip section and a brush holding section before providing a brush section;

FIG. 3 is a view of a brush piece viewed in the central axis line direction;

FIG. 4 is a cross-sectional view of a pair of brush pieces having a concave-to-concave structure, including the central axis;

FIG. 5 is a cross-sectional view of the brush holding section taken along line V-V of FIG. 2 including a locking section as viewed in the central axis line direction;

FIG. 6 is a view of a state where a locking section is locked to an inner peripheral surface of a brush piece;

FIG. 7 is a cross-sectional view of a brush piece including a convex section formed on a side surface of an annular section, including the central axis line;

FIG. 8 is a view of a state where a locking section is locked to a inner peripheral surface of a brush piece in a nasal cavity cleaner according to a second embodiment;

FIG. 9 is a view of a brush holding section including a locking section in a nasal cavity cleaner according to a third embodiment, viewed in the central axis line direction;

FIG. 10 is a view of a brush holding section including a locking section in a nasal cavity cleaner according to a fourth embodiment, viewed in the central axis line direction;

FIG. 11 is a view of a state where a locking section is locked to an inner peripheral surface of a brush piece in a nasal cavity cleaner according to a fifth embodiment; and

FIG. 12 is a view of a state where a locking section is locked to an inner peripheral surface of a brush piece in a nasal cavity cleaner according to a sixth embodiment.

### DESCRIPTION OF THE EMBODIMENTS

Exemplary embodiments will be described below with reference to the accompanying drawings. It should be noted that same reference numerals are given to the same or corresponding parts in each figure, and overlapping explanations will be omitted. Further, as will be described later, nasal cavity cleaners 100 to 100E will be exemplified below, but a configuration provided by each of these nasal cavity cleaners 100 to 100E is the same as a configuration provided by each of oral cavity cleaners 200 to 200E. In other words, in the following description, each of the nasal cavity cleaners 100 to 100E can be read as each of the corresponding oral cavity cleaners 200 to 200E.

### First Embodiment

### [Overall Configuration]

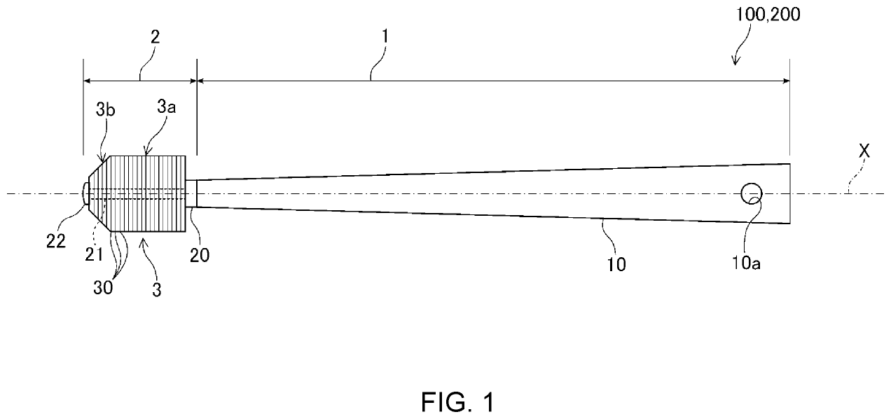

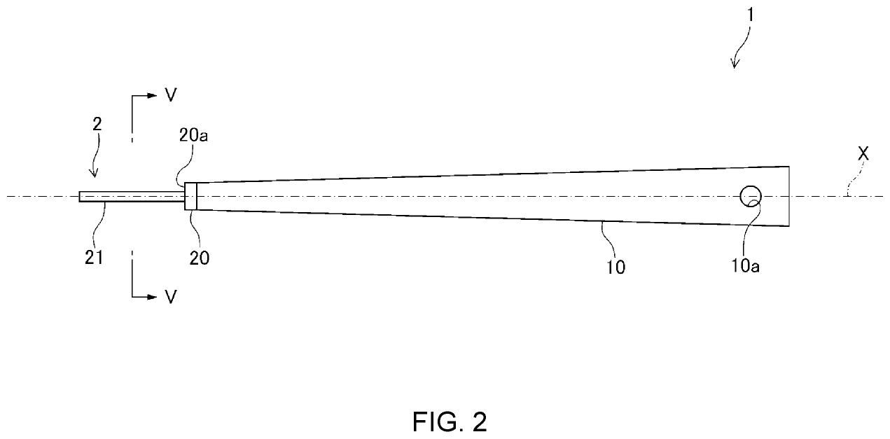

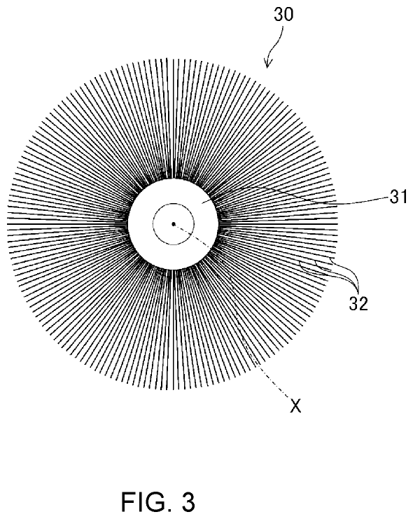

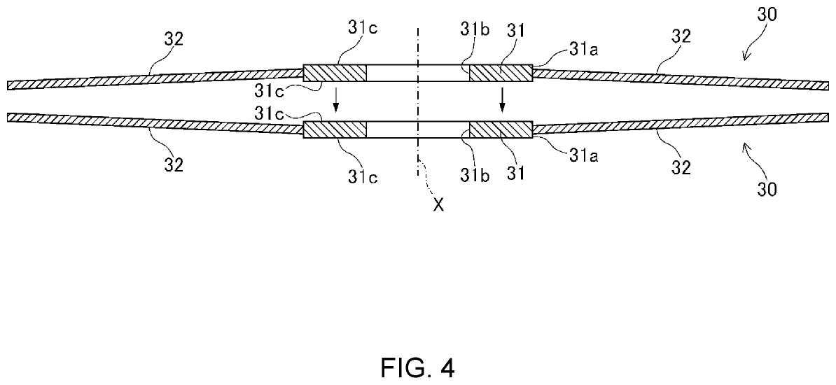

FIG. 1 is a view of a nasal cavity cleaner 100 according to a first embodiment. FIG. 2 is a view of a grip section 1 and a brush holding section 2 before a brush section 3 is provided. FIG. 3 is a view of brush pieces 30 viewed in the central axis line X direction. FIG. 4 is a cross-sectional view of a pair of the brush pieces 30 having a concave-to-concave structure, including the central axis line X. In the following description, the “central axis line” refers to an imaginary line passing through the center of the brush holding section 2 of the nasal cavity cleaner 100. In addition, the central axis line X in the brush section 3 before attached to the brush holding section 2 is an imaginary line passing through the center of the brush holding section 2 when the brush section 3 is attached to the brush holding section 2.

As shown in FIGS. 1 to 4, the nasal cavity cleaner 100 is an instrument to be inserted into the nasal cavity for use of cleaning the nasal cavity. Specifically, the nasal cavity cleaner 100 is configured as a so-called 360-degree brush to be inserted into the nasal cavity and then rotated, thereby enabling removal of foreign substances accumulated in the nasal cavity. The “nasal cavity” refers to the internal space of the nose, and may be, for example, the space from the nostril (that is, the opening corresponding to the air inlet and outlet) to the nasal turbinate (that is, the folds at the back of the nose). “Foreign substances” may refer to secretions from the nasal cavity or intruding objects from the outside to be removed from the nasal cavity. Secretions from the nasal cavity may be, for example, nasal discharge. In addition, intruding objects from the outside may be, for example, various allergens that can cause allergic reactions. The nasal cavity cleaner 100 may also be referred to as a nasal brush, a nasal cavity brush, or the like, or may be referred to as a nasal washer, a nasal cavity washer, or the like.

First, an overall configuration of the nasal cavity cleaner 100 will be described. The nasal cavity cleaner 100 includes the grip section 1, the brush holding section 2 and the brush section 3.

The grip section 1 is a part to be gripped by a user. The “user” refers to a person who operates the nasal cavity cleaner 100. This person may be a person having a nasal cavity to be cleaned with the nasal cavity cleaner 100 or another person other than the person having a nasal cavity to be cleaned with the nasal cavity cleaner 100. The grip section 1 includes a main body section 10.

The main body section 10 is a part to be held by the user. The main body section 10 is an elongated member extending along the central axis line X. From the viewpoint of operability of the nasal cavity cleaner 100, the main body section 10 may preferably have a length of 8 cm or longer and 18 cm or shorter, more preferably 10 cm or longer and 13 cm or shorter, and even more preferably 11 cm or longer and 12 cm or shorter. The main body section 10 may be made, for example, of hard plastic. The main body section 10 may be formed, for example, by injection molding.

Here, the main body section 10 extends linearly along the central axis line X and has a circular cross-section perpendicular to the central axis line X. As a result, the user can rotate the nasal cavity cleaner 100 around the central axis line X by reciprocating his or her fingers in a direction perpendicular to the central axis line X while holding the main body section 10 in his or her hand. In other words, the main body section 10 is formed to be rotatable in the user's hand. The main body section 10 is reduced in diameter toward a tip end of the nasal cavity cleaner 100 (that is, toward an end on the side of the brush holding section 2 and the brush section 3 opposite to the grip section 1 in the central axis line X direction). In other words, the main body section 10 is in the shape of a truncated cone reduced in diameter toward the tip end of the nasal cavity cleaner 100.

The main body section 10 has a through-hole 10a penetrating in the direction perpendicular to the central axis line X near a base end of the nasal cavity cleaner 100 (that is, near an end on the grip section 1 side opposite to the brush holding section 2 and the brush section 3, or on the side opposite to the tip end in the direction of the central axis line X). The through-hole 10a may be used, for example, for passing a string or the like therethrough.

The brush holding section 2 is a shaft-shaped part extending from the main body section 10 of the grip section 1. The brush holding section 2 is inserted through (or to be accurate, inserted into and passed through) the brush piece 30, which will be described later, thereby holding the brush piece 30. The brush holding section 2 may be formed integrally with the grip section 1, or may be formed separately from the grip section 1 and then connected to the grip section 1 by any method. The brush holding section 2 includes a base end block section 20, a shaft section 21 and a pressurizing lid section 22.

The base end block section 20 is a part provided adjacent to the main body section 10 of the grip section 1. The base end block section 20 may be made, for example, of hard plastic. The base end block section 20 may be formed, for example, by injection molding. The base end block section 20 includes a wall section 20a that is a surface perpendicular to the central axis line X on the tip end side of the nasal cavity cleaner 100. The wall section 20a is the surface on which an annular section 31 of the brush piece 30 abuts when the brush holding section 2 is first inserted though the annular section 31. This will be described later in detail. Here, the wall section 20a is configured as the surface perpendicular to the central axis line X.

The shaft section 21 is a part inserted through the brush piece 30. In other words, the shaft section 21 is a part functioning as the central axis of the brush section 3 when the brush section 3 is composed of a plurality of the brush pieces 30 through which the shaft section 21 is inserted. The shaft section 21 is an elongated part extending along the central axis line X. The shaft section 21 is formed to be smaller in diameter than the base end block section 20. The shaft section 21 has the same diameter over its entire length from the end of the shaft section 21 on the base end side of the nasal cavity cleaner 100 to the end of the shaft section 21 on the tip end side of the nasal cavity cleaner 100. The shaft section 21 may be made, for example, of hard plastic. The shaft section 21 may be formed, for example, by injection molding.

On one hand, the shaft section 21 is provided with no specific configuration on the tip end before the brush section 3 is provided to the brush holding section 2 in the manufacturing process of the nasal cavity cleaner 100 (see FIG. 2). On the other hand, after the brush section 3 is provided to the brush holding section 2 in the manufacturing process of the nasal cavity cleaner 100, the shaft section 21 is crushed at the end on the tip end side of the nasal cavity cleaner 100 to form a lid section to form a lid part.

The pressurizing lid section 22, which is the above-mentioned lid part, is fixed while pressurizing the brush pieces 30 of the brush section 3 toward the grip section 1 side. That is to say, the pressurizing lid section 22 pressurizes and thus holds the annular section 31 of the brush piece 30 along the central axis line X toward the grip section 1 side when the brush holding section 2 is last inserted through the annular section 31. This will be described later in detail. The pressurizing lid section 22 is formed by applying ultrasonic welding from the tip end side of the nasal cavity cleaner 100 after inserting the brush holding section 2 through all the brush pieces 30. Specifically, the pressurizing lid section 22 is formed by bringing a curved concave member into contact with the tip end of the shaft section 21 to melt the shaft section 21. The pressurizing lid section 22 is larger in outer diameter than the shaft section 21. The pressurizing lid section 22 may be larger in outer diameter than the shaft section 21 by 0.5 mm or more and 2 mm or less, 0.7 mm or more and 1.5 mm or less, or 0.9 mm or more and 1.2 mm or less. On one hand, the larger in outer diameter the pressurizing lid section 22 is as compared to the shaft section 21, the less likely the brush pieces 30 are to come off and rotate idly. On the other hand, the smaller in outer diameter the pressurizing lid section 22 is, the less uncomfortable feeling such as being caught in the nasal cavity there is in the nasal cavity.

The brush section 3 is a part to be inserted into the nasal cavity for cleaning the nasal cavity. The brush section 3 is a so-called 360-degree brush part. Specifically, the brush section 3 is configured in a manner that a large number of brush bristles 32 of the brush piece 30 are provided radially outward in the entire circumferential direction around the central axis line X. From the viewpoint of efficiency of cleaning the nasal cavity, the brush section 3 may be preferably provided with 10,000 or more brush bristles 32, more preferably 30,000 or more brush bristles 32, and even more preferably 60,000 or more brush bristles 32. Here, the brush section 3 is provided with 70,000 brush bristles 32. The greater number of the brush bristles 32 the brush section 3 is provided with (that is, the higher the density is), the better the ability to clean the nasal cavity is improved. Therefore, it is preferable to increase the number of the brush bristles 32 (that is, to increase the density). It should be noted that, from the viewpoint of efficiency of cleaning the nasal cavity, the number of brush bristles 32 provided to the brush section 3 may be 100,000 or more, while from the viewpoint of facilitating washing-off of foreign substances adhering to the brush section 3, the number of the brush bristles 32 provided to the brush section 3 may be 100,000 or less.

The brush section 3 includes a plurality of brush pieces 30 stacked on each other and held by the brush holding section 2. Further, the brush section 3 includes a same-diameter section 3a and a reduced-diameter section 3b in its overall shape. The same-diameter section 3a is a part having a cylindrical outer shape with a same diameter along the central axis line X direction. On one hand, each of the brush pieces 30 constituting the-same diameter section 3a has a same outer diameter such as any numerical value of 10 mm or larger and 21 mm or smaller, 16 mm or larger and 20 mm or smaller, and 17 mm or larger and 19 mm or smaller. On the other hand, the reduced-diameter section 3b is a part being a truncated cone in outer shape reduced in diameter toward the tip end of the nasal cavity cleaner 100 along the central axis line X direction. Of the plurality of brush pieces 30 constituting the reduced-diameter section 3b, the brush piece 30 closest to the tip end of the nasal cavity cleaner 100 has an outer diameter smaller than the other brush pieces 30 such as an outer diameter of any numerical value of 7 mm or more and 14 mm or less, 10.5 mm or more and 13.5 mm or less, and 11 mm or more and 13 mm or less. On the cross-section including the central axis line X, the angle between the outer shape of the reduced-diameter section 3b (that is, the envelope of tip ends of the brush bristles 32) and the central axis line X may be 35 degrees or more and 50 degrees or less, or 40 degrees or more and 45 degrees or less.

Each of the brush pieces 30 includes the annular section 31 and a plurality of brush bristles 32. On one hand, the annular sections 31 of the brush pieces 30 are the same in shape and size. On the other hand, the brush bristles 32 of the brush pieces 30 are not necessarily the same in length, thereby realizing the same-diameter section 3a and the reduced-diameter section 3b.

The annular section 31 is the central part of the brush piece 30. The annular section 31 is formed in annular shape. While “annular” means “of a closed ring” including “of a circular ring”, “of a rectangular ring” and “of a polygonal ring”, the annular section 31 is formed specifically in the shape of circular ring. More specifically, the annular section 31 is formed annularly so that at least an inner peripheral surface 31b is circular in shape when viewed in the central axis line X direction, and here, an outer peripheral surface 31a is also circular in shape when viewed in the central axis line X direction. The annular section 31 may be larger in outer diameter than the pressurizing lid section 22. The annular section 31 may be made, for example, of nylon or polybutylene terephthalate resin. Each of the brush pieces 30 is held by the brush holding section 2 by inserting the shaft section 21 of the brush holding section 2 through the annular section 31.

The brush bristle 32 is a part for scraping out foreign substances in the nasal cavity and formed in the shape of flexible and elastic bristles. The brush bristle 32 is provided in plurality on the outer peripheral surface 31a of the annular section 31. Each of the brush bristles 32 is provided radially outward from the outer peripheral surface 31a of the annular section 31 over the entire circumference of the outer peripheral surface 31a of the annular section 31. In other words, the plurality of brush bristles 32 are provided on the outer peripheral surface 31a of the annular section 31 over 360 degrees. The brush bristle 32 may be made, for example, of nylon or polybutylene terephthalate resin. In this case, if the brush bristle 32 is immersed in hot water or the like, the flexibility of the brush bristle 32 is enhanced to improve the feeling of use. Each of the brush bristles 32 may be formed to be approximately 0.05 mm in thickness.

On one hand, the brush pieces 30 constituting the same-diameter section 3a are the same in length. On the other hand, the brush pieces 30 constituting the reduced-diameter section 3b are shorter in length toward the tip end side of the nasal cavity cleaner 100. In other words, at least at the end opposite to the grip section 1 (that is, at the end on the tip end side of the nasal cavity cleaner 100), the brush pieces 30 are formed to include the brush bristles 32 shorter in length from the grip section 1 side toward the side opposite to the grip section 1. In this manner, the brush pieces 30 constitute the reduced-diameter section 3b of the brush section 3.

The brush pieces 30 configured in this manner are held by the shaft section 21 of the brush holding section 2, stacked on each other so that positions of the annular sections 31 align with each other, or in other words, the shaft section 21 of the brush holding section 2 is inserted through annular sections 31. Each of the brush pieces 30 stacked on each other is in contact with another adjacent one of the brush pieces 30 on the side surfaces 31c of the annular sections 31 of the bush pieces adjacent to each other. It should be noted that the side surfaces 31c of the annular section 31 are the surfaces on both sides of the annular section 31 in the central axis line X direction.

In addition, each of the brush bristles 32 is provided with a slight inclination to the surface perpendicular to the central axis line X in either one of directions toward the grip section 1 side and toward the tip end side opposite to the grip section 1 when viewed in the central axis line X. In other words, the brush piece 30 is not perfectly planar in shape but slightly funnel-shaped. In addition, each two of the brush pieces 30 form a concave-to-concave structure in pairs. The “concave-to-concave structure” refers to a structure in which a pair of the brush pieces 30 are combined with each other so that concave sides of the funnel-shaped brush pieces 30 face each other (see FIG. 4). This enables the side surfaces 31c of the pair of the brush pieces 30 facing each other to contact each other, and tip ends of the brush bristles 32 to contact or cross each other. The brush section 3 is configured by stacking a large number of the pairs of the brush pieces 30 having the concave-to-concave structure on each other. As a consequence, when the nasal cavity cleaner 100 is used, foreign substances in the nasal cavity can be gathered in a mass and trapped in a gap between the concave-to-concave structures adjacent to each other, thereby improving the efficiency of cleaning the nasal cavity. Moreover, foreign substances are less likely to enter the roots of the brush bristles 32, thereby facilitating washing-off of foreign substances adhering to the brush section 3. In addition, while the brush bristles 32 used in the nasal cavity cleaner 100 are ultrafine, the concave-to-concave structure realizes the brush section 3 of sufficient stiffness even with the ultrafine brush bristles 32.

[Idle Rotation Suppression Structure]

Next, an idle rotation suppression structure for suppressing idle rotation of the brush holding section 2 in relation to the brush pieces 30 in the nasal cavity cleaner 100 will be described.

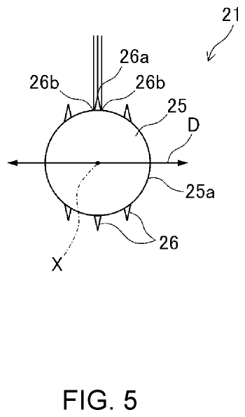

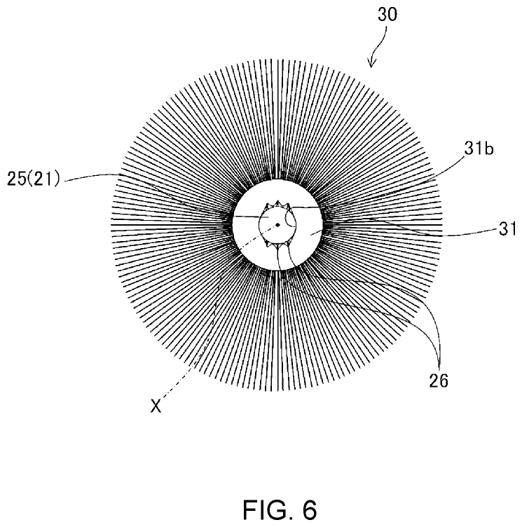

FIG. 5 is a cross-sectional view of the brush holding section 2 taken along line V-V of FIG. 2 including a locking section 26 as viewed in the central axis line X direction. FIG. 6 is a view of the locking section 26 locked to the inner peripheral surface 31b of the brush piece 30. As shown in FIGS. 5 and 6, the locking section 26 provided to the brush holding section 2 of the nasal cavity cleaner 100 suppresses idle rotation of the brush holding section 2 in relation to the brush piece 30. The brush holding section 2 includes a base section 25 and the locking section 26.

The base section 25 is a main part of the shaft section 21 of the brush holding section 2. The base section 25 has a cylindrical shape with an outer diameter equal to or slightly larger than the inner diameter of the annular section 31. On one hand, the outer diameter of the base section 25 may be larger than the inner diameter of the annular section 31 by, for example, 0 mm or more and 0.3 mm or less, 0.03 mm or more and 0.2 mm or less, or 0.05 mm or more and 0.1 mm or less. This suggests that even without any locking section 26 provided, the base section 25 is press-fitted into the annular section 31 while expanding the inner peripheral surface 31b of the annular section 31. On the other hand, the base section 25 may have a cylindrical shape with an outer diameter smaller than the inner diameter of the annular section 31. In this case, the base section 25 is loosely fitted into the annular section 31 of the brush piece 30 without any locking section 26 provided.

The locking section 26 is locked to the inner peripheral surface 31b of the annular section 31 of the brush piece 30 held by the brush holding section 2 (or by the shaft section 21), thereby suppressing the rotation of the brush piece 30 (namely, asynchronous rotation in relation to the brush holding section 2 around the central axis line X). “Locking” means that any structures are related to each other, and more specifically, that asynchronous rotation around the central axis line X above a certain level is suppressed. “Locking” may include at least one of, for example, hooking, biting into, pressurizing, and the like.

The locking section 26 is a rib-shaped structural part extending along the central axis line X of the brush holding section 2 on an outer peripheral surface 25a of the base section 25. Here, the brush holding section 2 includes six locking sections 26. The locking sections 26 are provided symmetrically in relation to a predetermined direction intersecting the central axis line X and perpendicular to the central axis line X (here, the reference direction D in FIG. 5). More particularly, the locking sections 26 are provided symmetrically in relation to a plane including the central axis line X and the reference direction D. The locking section 26 is formed so that an outer diameter from the central axis line X of the brush holding section 2 to a top 26a of the locking section 26 (namely, the highest part of the rib constituting the locking section 26) is larger than the inner diameter of the annular section 31. The outer diameter of the locking section 26 (namely, the outer diameter from the central axis line X to the top 26a) may be larger than the inner diameter of the annular section 31 by, for example, 0.05 mm or more and 0.6 mm or less, or 0.1 mm or more and 0.5 mm or less, or 0.15 mm or more and 0.4 mm or less. However, the outer diameter of the locking section 26 should take a value larger than the outer diameter of the base section 25.

The brush holding section 2 including such locking section 26 is inserted through the annular section 31 by press fitting, thereby locking the locking section 26 to the inner peripheral surface 31b of the annular section 31. The inner peripheral surface 31b of the annular section 31 of each of the brush pieces 30 is pressed and expanded by the locking section 26 while the brush holding section 2 is inserted through the annular section 31. In other words, the locking section 26 is formed to have a higher strength than the inner peripheral surface 31b of the annular section 31. Pressing and expanding the inner peripheral surface 31b of the annular section 31 by using the locking section 26 in this manner can suppress a rotational movement of the locking section 26 around the central axis line X due to a portion of the inner peripheral surface 31b left unpressed and unexpanded between the locking sections 26 adjacent to each other. Moreover, a repulsive force from the inner peripheral surface 31b pressed and expanded by the locking section 26 increases the frictional force acting between the locking section 26 and the inner peripheral surface 31b, enabling further suppression of the rotational movement of the locking section 26 around the central axis line X.

It should be noted that on the cross-section perpendicular to the central axis line X of the brush holding section 2, the locking section 26 is formed so that the top 26a of the locking section 26a is positioned between two interfaces 26b between the locking section 26 and the base section 25 when viewed in at least one direction perpendicular to the central axis line X (here, in the reference direction D in FIG. 5). Refer to the positional relationship of three parallel lines shown in FIG. 5. In other words, on the cross-section perpendicular to the central axis line X of the brush holding section 2, one of the interfaces 26b, the top 26a and the other of the interfaces 26b are arranged in this order when viewed in the reference direction D. Here, the “interface” is a boundary between the base section 25 and the locking section 26, found on both sides of each of the locking sections 26. Therefore, to sum up, of the three parallel lines shown in FIG. 5, the part indicated by the central line is the top 26a, and the parts indicated by the left and right lines are the interfaces 26b. And, at least one reference direction such as the reference direction D in FIG. 5 in which one of the interfaces 26b, the top part 26a, and the other of the interfaces 26b are arranged in this order is found. It should be noted that when viewed in the reference direction D on the cross-section perpendicular to the central axis line X of the brush holding section 2, the locking section 26 has a simple inclination from one of the interfaces 26b toward the top 26a (that is, an inclination to only one of the left and right sides along the reference direction D in FIG. 5). Further, the locking section 26 has a simple inclination from the top 26a toward the other of the interfaces 26b when viewed in the reference direction D on the cross-section perpendicular to the central axis line X of the brush holding section 2 (that is, an inclination to only the other of the left and right sides along the reference direction D in FIG. 5). In other words, the locking section 26 has a so-called draft angle facilitating the removal of the brush holding section 2 from a mold when manufacturing the brush holding section, for example, by injection molding.

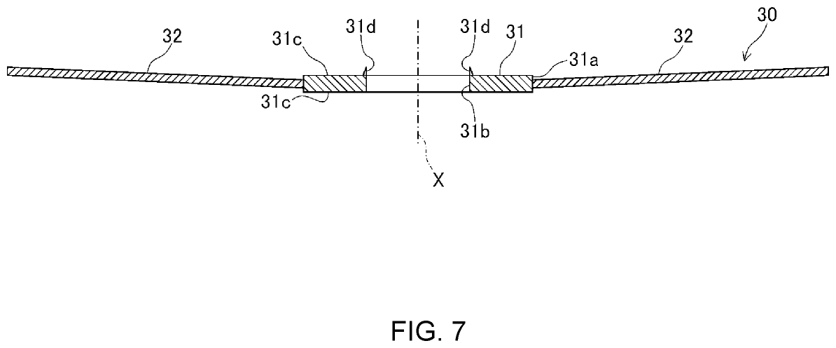

FIG. 7 is the cross-sectional view of the brush piece 30 including a convex section 31d formed on the side surface 31c of the annular section 31, including the central axis line X. As shown in FIG. 7, the convex section 31d is formed on the side surface 31c of the annular section 31 of each brush piece 30. Therefore, between each of the brush pieces 30 and another adjacent one of the brush pieces 30 stacked on each other, the convex section 31d formed on the side surface 31c of the annular section 31 (or in other words, an increase in frictional force due to the convex section 31d) suppresses relative rotation around the central axis line X. It should be noted that while in FIG. 7, the convex section 31d is formed only on one side surface 31c of the annular section 31, the convex section 31d may be formed on both side surfaces 31c of the annular section 31. The convex section 31d may be a burr formed in the manufacturing process of the brush pieces 30, for example.

The shaft section 21 of the brush holding section 2 is inserted through each of the brush pieces 30. Thus, the brush pieces 30 are stacked on each other to configure the brush section 3. At this time, when the shaft section 21 of the brush holding section 2 is first inserted through the annular section 31 of the brush piece 30, the annular section 31 abuts the wall section 20a of the base end block section 20. Further, when the shaft section 21 of the brush holding section 2 is last inserted through the annular section 31 of the brush piece 30, the annular section 31 is pressurized along the central axis line X toward the grip section 1 side and held by the pressurizing lid section 22. As a result, a frictional force is generated between the side surfaces 31c of the annular sections 31 of the brush pieces 30 adjacent to each other. Further, when it comes to the brush pieces 30 at both ends, a frictional force is generated between the side surface 31c of the annular section 31 and the wall section 20a of the base end block section 20 or the pressurizing lid section 22. As a result, the plurality of brush pieces 30 are cooperatively locked to the brush holding section 2.

[Functions and Effects]

As described above, the nasal cavity cleaner 100 is an instrument to be inserted into the nasal cavity for cleaning the nasal cavity. The nasal cavity cleaner 100 includes the grip section 1 to be gripped by a user, the shaft-shaped brush holding section 2 extending from the grip section 1, and the plurality of brush pieces 30 stacked on each other and held by the brush holding section 2. Each of the plurality of brush pieces 30 includes the annular section 31 formed in an annular shape and the plurality of brush bristles 32 provided on the outer peripheral surface 31a of the annular section 31. The shaft section 21 of the brush holding section 2 is inserted through the annular section 31, whereby each of the plurality of brush pieces 30 is held by the brush holding section 2. The brush holding section 2 includes the locking section 26 locked to the inner peripheral surface 31b of the annular section 31 of the brush piece 30 held by the brush holding section 2, thereby suppressing the rotation of the brush piece 30 around the central axis line X.

According to the nasal cavity cleaner 100, the shaft-shaped brush holding section 2 is inserted through the annular sections 31 of the plurality of brush pieces 30 stacked on each other, so that the brush section 3 to be inserted into the nasal cavity is configured. Here, the brush holding section 2 is provided with the locking section 26 locked to the annular section 31 of the brush piece 30. Therefore, when the oral cavity cleaner 100 is rotated around the central axis line X in the oral cavity, the friction between the inner wall of the oral cavity and the brush bristles 32 can suppress the failure of the brush holding section 2 to hold the brush pieces 30, enabling the brush pieces 30 to rotate integrally with the brush holding section 2. This enables the suppression of idle rotation of the brush holding section 2 in relation to the brush pieces 30. Moreover, the nasal cavity cleaner 100 can easily achieve a large amount of brush bristles 32 that is hard to be achieved by conventionally known twisted brushes and flocked brushes. On one hand, the twisted brush is a brush including twisted wires or other members as a main shaft and a brush section formed by pinching brush bristles when twisting the wires or the like. On the other hand, the flocked brush is a brush including a brush section formed by implanting bristles on a main shaft. Despite the difficulty in increasing the amount of bristles, these twisted brush and flocked brush have no problem of idle rotation of the brush holding section 2 in relation to the brush pieces 30 as the present disclosure generally has.

In the nasal cavity cleaner 100, the plurality of brush bristles 32 are provided radially outward from the outer peripheral surface 31a of the annular section 31 over the entire circumference of the outer peripheral surface 31a of the annular section 31. This enables effective removal of foreign substances in the nasal cavity by rotating the nasal cavity cleaner around the central axis line X while the brush section 3 is inserted into the nasal cavity.

In the nasal cavity cleaner 100, the annular section 31 is formed in an annular shape. The brush holding section 2 includes the cylindrical base section 25 and the rib-shaped locking section 26 extending on the outer peripheral surface 25a of the cylindrical base section 25 along the central axis line X of the brush holding section 2 and having an outer diameter from the central axis line X of the brush holding section 2 to the top 26a larger than the inner diameter of the annular section 31. The brush holding section 2 is inserted through the annular section 31 by press fitting, thereby locking the locking section 26 to the inner peripheral surface 31b of the annular section 31. This enables the brush piece 30 to be locked to the brush holding section 2 more firmly, thereby more reliably suppressing the idle rotation of the brush holding section 2 in relation to the brush pieces 30.

In the nasal cavity cleaner 100, on the cross-section perpendicular to the central axis line X of the brush holding section 2, the top 26a of the locking section 26a is positioned between the two interfaces 26b between the locking section 26 and the base section 25 when viewed in at least one direction perpendicular to the central axis line X (here, in the reference direction D in FIG. 5). This facilitates the removal of the brush holding section 2 from the mold when manufacturing the brush holding section 2, for example, by injection molding.

In the nasal cavity cleaner 100, the inner peripheral surface 31b of the annular section 31 of each of the brush pieces 30 is pressed and expanded by the locking section 26 while the brush holding section 2 is inserted through the annular section 31. This facilitates the manufacturing of the brush piece 30 as there is no need of forming in advance a recess corresponding to the locking section 26 on the inner peripheral surface 31b of the annular section 31. Moreover, a pressing force generated between the locking section 26 and the inner peripheral surface 31b of the annular section 31 can lock the brush pieces 30 more firmly to the brush holding section 2, thereby enabling more reliable suppression of idle rotation of the brush holding section 2 in relation to the brush piece 30.

In the nasal cavity cleaner 100, each of the plurality of brush pieces 30 stacked on each other is in contact with another adjacent one of the brush pieces 30 on the side surfaces 31c of the annular sections 31 of the bush pieces adjacent to each other. According to this, a frictional force generated between the side surfaces 31c of the annular sections 31 of the brush pieces 30 adjacent to each other suppresses the relative rotation of the brush pieces 30 around the central axis line X. As a result, the plurality of brush pieces 30 are locked to the brush holding section 2 in cooperation with each other, thereby enabling more reliable suppression of idle rotation of the brush holding section 2 in relation to the brush pieces 30.

In the nasal cavity cleaner 100, each of the plurality of brush pieces 30 includes the convex section 31d formed on the side surface 31c of the annular section 31. According to this, the side surfaces 31c of the annular sections 31 of the brush pieces 30 adjacent to each other are firmly connected to each other by the convex sections 31d, thereby suppressing the relative rotation of the brush pieces 30 around the central axis line X. As a result, the plurality of brush pieces 30 are locked to the brush holding section 2 in cooperation with each other, thereby enabling more reliable suppression of idle rotation of the brush holding section 2 in relation to the brush pieces 30.

In the nasal cavity cleaner 100, the brush holding section 2 includes the wall section 20a of the base end block section 20 on which the annular section 31 of the brush piece 30 abuts when the brush holding section 2 is first inserted through the annular section 31, and the pressurizing lid section 22 pressurizing and holding the annular section 31 of the brush piece 30 along the central axis line X toward the grip section 1 side when the brush holding section 2 is last inserted through the annular section 31. According to this, the plurality of brush pieces 30 are pressurized and held by the wall section 20a and the pressurizing lid section 22 of the brush holding section 2, thereby enabling more reliable suppression of idle rotation of the brush holding section 2 in relation to the brush pieces 30.

In the nasal cavity cleaner 100, the pressurizing lid section 22 is formed by ultrasonic welding. This facilitates the manufacturing of the nasal cavity cleaner 100 provided with the above-described configuration.

In the nasal cavity cleaner 100, the plurality of brush pieces 30 are formed to include the brush bristles 32 shorter in length from the grip section 1 side toward the side opposite to the grip section 1 at least at the end opposite to the grip section 1. According to this, the brush section 3 is in a tapered shape, thereby facilitating the insertion of the brush section 3 into the nasal cavity. Furthermore, when the brush piece 30 positioned at the end opposite to the grip section 1 is supported by a small-diameter lid section, it is hard to ensure the frictional force between the side surface 31c of the annular section 31 of the brush piece 30 and the lid section. In this case, shortening of the brush bristles 32 of the brush piece 30 in length reduces a torque acting on the brush piece 30 owing to the friction between the inner wall of the nasal cavity and the brush bristles 32, thereby enabling the suppression of the excess of this torque over the frictional force between the side surface 31c of the annular section 31 and the lid section. This enables more reliable suppression of the idle rotation of the brush holding section 2 in relation to the brush pieces 30.

### Second Embodiment

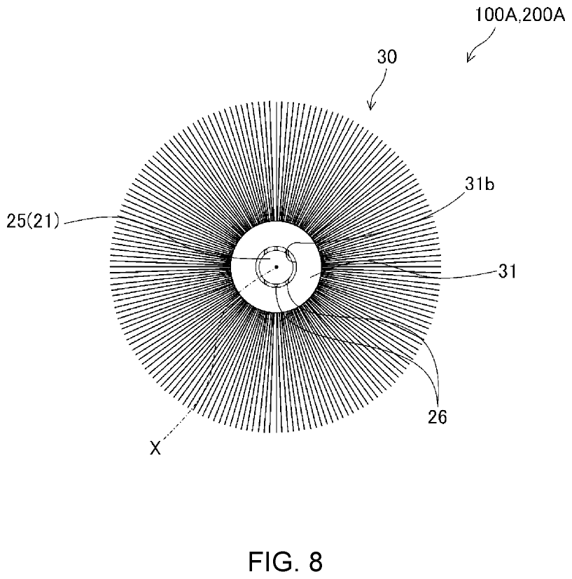

FIG. 8 is a view of a state where the locking section 26 is locked to the inner peripheral surface 31b of the brush piece 30 in a nasal cavity cleaner 100A according to the second embodiment. As shown in FIG. 8, as compared to the nasal cavity cleaner 100 according to the first embodiment, the nasal cavity cleaner 100A according to the second embodiment differs mainly in the state of a contact between the inner peripheral surface 31b of the brush piece 30 and the locking section 26 and is common thereto in other respects.

In the nasal cavity cleaner 100A, the locking section 26 is crushed by the inner peripheral surface 31b of the annular section 31 in the state where the brush holding section 2 is inserted through the annular section 31. That is, the locking section 26 is formed to have a lower strength than the inner peripheral surface 31b of the annular section 31. The locking section 26 is crushed by the inner peripheral surface 31b of the annular section 31 in this manner, so that a repulsive force from the crushed locking section 26 causes increase in the frictional force acting between the locking section 26 and the inner peripheral surface 31b and enables even better suppression of the rotational movement of the locking section 26 around the central axis line X. In addition, when the brush holding section 2 is inserted through the annular section 31, the locking section 26 may press and expand the inner peripheral surface 31b of the annular section 31, and at the same time, the inner peripheral surface 31b of the annular section 31 may crush the locking section 26.

In this manner, in the nasal cavity cleaner 100A, the locking section 26 is crushed by the inner peripheral surface 31b of the annular section 31 in the state where the brush holding section 2 is inserted through the annular section 31. This facilitates the manufacturing of the brush piece 30 as there is no need of forming in advance a recess corresponding to the locking section 26 on the inner peripheral surface 31b of the annular section 31. Moreover, a pressing force generated between the locking section 26 and the inner peripheral surface 31b of the annular section 31 can lock the brush pieces 30 more firmly to the brush holding section 2, thereby enabling more reliable suppression of idle rotation of the brush holding section 2 in relation to the brush piece 30.

### Third Embodiment

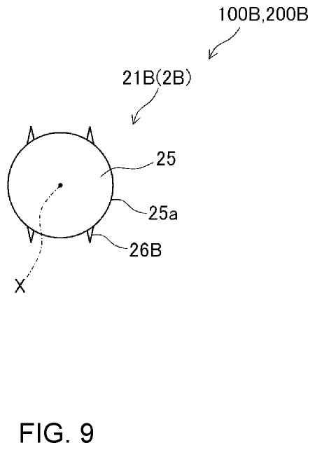

FIG. 9 is a view of a brush holding section 2B including a locking section 26B in a nasal cavity cleaner 100B according to the third embodiment, viewed in the central axis line X direction. As shown in FIG. 9, the nasal cavity cleaner 100B according to the third embodiment differs mainly in the number of locking sections 26B provided to a shaft section 21B as compared to the nasal cavity cleaner 100 according to the first embodiment or the nasal cavity cleaner 100A according to the second embodiment, and is common thereto in other respects.

Four locking sections 26B are provided on the outer peripheral surface 25a of the base section 25 in the nasal cavity cleaner 100B. In other words, the brush holding section 2B includes four locking sections 26B. In this regard, the brush holding section 2B may include other than four (or six) locking sections 26B.

### Fourth Embodiment

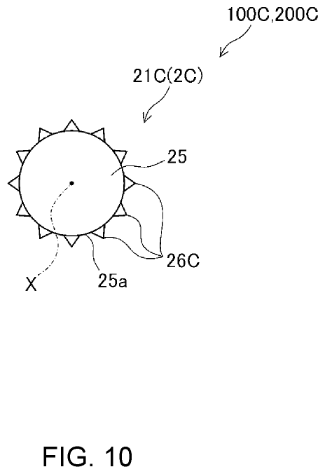

FIG. 10 is a view of a brush holding section 2C including a locking section 26C in a nasal cavity cleaner 100C according to the fourth embodiment, viewed in the central axis line X direction. As shown in FIG. 10, the nasal cavity cleaner 100C according to the fourth embodiment differs mainly in the direction in which the top of the locking section 26C faces as compared to the nasal cavity cleaner 100 according to the first embodiment or the nasal cavity cleaner 100A according to the second embodiment, and is common thereto in other respects.

In the nasal cavity cleaner 100C, on the cross-section perpendicular to the central axis line X of the brush holding section 2C, when viewed in at least one direction perpendicular to the central axis line X, the locking section 26c is not formed so that the top of the locking section 26C is positioned between two interfaces between the locking section 26C and the base section 25. It should be noted that in FIG. 10, the locking sections 26C are provided at point-symmetrical positions around the central axis line X.

### Fifth Embodiment

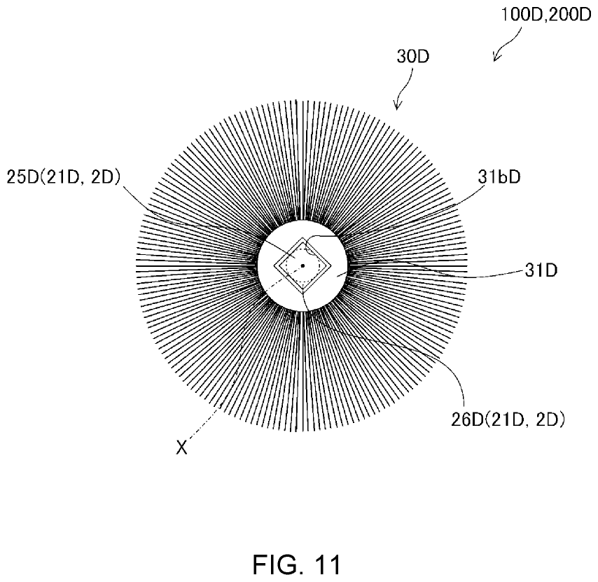

FIG. 11 is a view of a state where a locking section 26D is locked to an inner peripheral surface 31bD of a brush piece 30D in a nasal cavity cleaner 100D according to the fifth embodiment. As shown in FIG. 11, the nasal cavity cleaner 100D according to the fifth embodiment differs in the shape of an annular section 31D of the brush piece 30D and the shape of a shaft section 21D of a brush holding section 2D as compared to the nasal cavity cleaner 100 according to the first embodiment, and is common thereto in other respects.

In the nasal cavity cleaner 100D, the annular section 31D is formed in a polygonal annular shape so that at least the inner peripheral surface 31bD is in a polygonal shape. Here, “polygonal annular” refers to “of a ring having an inner circumference in a polygonal shape”, while an outer circumference may be polygonal or circular in shape. Specifically, the inner peripheral surface 31bD of the annular section 31D has a quadrangular shape. Moreover, in the nasal cavity cleaner 100D, the shaft section 21D of the brush holding section 2D is formed in a polygonal columnar shape so as to fit into the annular section 31D. Specifically, the shaft section 21D of the brush holding section 2D has a quadrangular prism shape. The shaft section 21D of the brush holding section 2D is loosely fitted into the annular section 31D. In other words, the shaft section 21D of the brush holding section 2D is formed to have a cross-section smaller than the annular section 31D. When the shaft section 21D of the brush holding section 2D and the annular section 31D are arranged coaxially (that is, when a circumferential gap is arranged so as to be equal), the size of the gap may be, for example, 0.01 mm or more and 0.3 mm or less, 0.05 mm or more and 0.2 mm or less, or 0.08 mm or more and 0.1 mm or less. On one hand, the smaller the gap size is, the smaller the backlash is and the less water or the like enters the gap. On the other hand, the larger the gap is, the easier the manufacturing is.

The shaft section 21D includes a base section 25D and the locking section 26D. The base section 25D corresponds to a central cylindrical part of the shaft section 21D. The locking section 26D corresponds to four corners on the cross-section (here, in a quadrangular shape) perpendicular to the central axis line X of the brush holding section 2D. In other words, the brush holding section 2D has a polygonal shape (here, a quadrangular shape) when viewed in the direction of the central axis line X, and the corners of the brush holding section 2D are regarded as the locking sections 26D.

As described above, in the nasal cavity cleaner 100D, at least the inner peripheral surface 31bD of the annular section 31D is formed in a polygonal columnar shape (here, in a quadrangular prism shape), and the locking section 26D corresponds to the corners of the cross-section perpendicular to the central axis line X of the brush holding section 2D. According to this, the shapes of the locking section 26D and the inner peripheral surface 31bD of the annular section 31D match each other, thereby enabling more reliable suppression of the idle rotation of the brush holding section 2D in relation to the brush pieces 30D.

In the nasal cavity cleaner 100D, the brush holding section 2D is loosely fitted into the annular section 31D. This facilitates the insertion of the brush holding section 2D through the annular section 31D.

### Sixth Embodiment

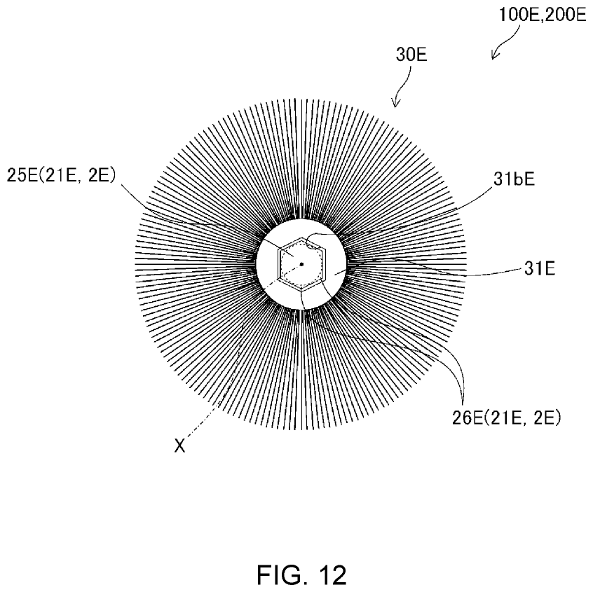

FIG. 12 is a view of a state where a locking section 26E is locked to an inner peripheral surface 31bE of a brush piece 30E in a nasal cavity cleaner 100E according to the sixth embodiment. As shown in FIG. 12, as compared to the nasal cavity cleaner 100D according to the fifth embodiment, the nasal cavity cleaner 100E according to the sixth embodiment differs mainly in the number of the locking sections 26E provided on a shaft section 21E of the brush holding section 2E and in the shape of the inner peripheral surface 31bE of the annular section 31E corresponding to the locking section 26E, and is common thereto in other respects.

In the nasal cavity cleaner 100E, the annular section 31E is formed in a polygonal annular shape so that at least the inner peripheral surface 31bE has a polygonal shape. Specifically, the inner peripheral surface 31bE of the annular section 31E has a hexagonal shape. Further, in the nasal cavity cleaner 100E, the shaft section 21E of the brush holding section 2E is formed in a polygonal columnar shape fitted into the annular section 31E. Specifically, the shaft section 21E of the brush holding section 2E has a hexagonal columnar shape. The shaft section 21E of the brush holding section 2E is loosely fitted into the annular section 31E.

Here, the shaft section 21E includes a base section 25E and the locking section 26E. On one hand, the base section 25E corresponds to a central cylindrical part of the shaft section 21E. On the other hand, the locking section 26E corresponds to six corners of the cross-section (here, in a hexagonal shape) perpendicular to the central axis line X of the brush holding section 2E. In other words, the brush holding section 2E has a polygonal shape (here, a hexagonal shape) when viewed in the direction of the central axis line X, and the corners of the brush holding section 2E are regarded as locking sections 26E.

[Modifications]

Each embodiment described above can be implemented in various forms modified or improved based on the knowledge of those skilled in the art.

For instance, in each of the above-described embodiments, a structure included in any one of the nasal cavity cleaners 100-100E may be partially combined with a structure included in the other nasal cavity cleaners 100-100E.

Also, in each of the above-described embodiments, the nasal cavity cleaners 100 to 100E are assumed to be used for human nasal cavities. However, the nasal cavity cleaners 100-100E may be used for the nasal cavities of non-human animals. In this case, the size of the brush section 3 (for example, the length of the brush section 3 in the central axis line X direction, the outer diameter of the brush section 3, the length of the brush bristles 32, etc.) should be preferably optimized for the nasal cavity of the animals.

Moreover, in each of the above-described embodiments, in the nasal cavity cleaners 100 to 100E, the reduced-diameter section 3b is a truncated cone in outer shape reduced in diameter toward the tip end of the nasal cavity cleaner 100 along the central axis line X direction. However, the reduced-diameter section 3b may have a dome shape in which an inclined part is configured with a curved surface.

Furthermore, in each of the above-described embodiments, in the nasal cavity cleaners 100 to 100E, the pressurizing lid section 22 is formed by ultrasonic welding. However, the pressurizing lid section 22 may be formed by a method other than ultrasonic welding.

Also, in each of the above-described embodiments, the nasal cavity cleaners 100 to 100E are assumed to be used for cleaning the nasal cavity. However, the nasal cavity cleaners 100 to 100E may be used for purposes such as cleaning teeth as well as the nasal cavity. In this case, the nasal cavity cleaners 100 to 100E may be formally referred to as oral cleaners, oral brushes, toothbrushes, or the like, or may simply be referred to as cleaners, brushes, or the like.

What is claimed is:

1. A nasal cavity cleaner to be inserted into a nasal cavity for cleaning the nasal cavity, the nasal cavity cleaner comprising:
a grip section to be gripped by a user;
a shaft-shaped brush holding section extending from the grip section; and
a plurality of brush pieces stacked on each other and held by the brush holding section,
wherein each of the plurality of brush pieces comprises:
an annular section formed in an annular shape; and
a plurality of brush bristles provided on an outer peripheral surface of the annular section, each of the plurality of brush pieces held by the brush holding section inserted through the annular section,
wherein the brush holding section comprises:
a base section in a cylindrical shape; and
a locking section extending on an outer peripheral surface of the base section along a central axis line of the brush holding section, the locking section formed in a rib shape having an outer diameter from the central axis line of the brush holding section to a top of the locking section, the outer diameter larger than an inner diameter of the annular section, and the locking section locked to an inner peripheral surface of the annular section of the brush piece held by the brush holding section to suppress a rotation of the brush piece around the central axis line,
wherein the brush holding section is inserted through the annular section by press fitting to lock the locking section to the inner peripheral surface of the annular section, and
wherein the locking section is crushed by the inner peripheral surface of the annular section with the brush holding section being inserted through the annular section.

2. The nasal cavity cleaner according to claim 1, wherein the plurality of brush bristles are provided radially outward from the outer peripheral surface of the annular section over an entire circumference of the outer peripheral surface of the annular section.

3. The nasal cleaner according to claim 1, wherein on a cross-section perpendicular to the central axis line of the brush holding section, the top of the locking section is positioned between two interfaces between the locking section and the base section when viewed in at least one direction perpendicular to the central axis line.

4. The nasal cleaner according to claim 1, wherein the inner peripheral surface of the annular section of each of the plurality of brush pieces is pressed and expanded by the locking section with the brush holding section being inserted through the annular section.

5. The nasal cleaner according to claim 1, wherein each of the plurality of brush pieces stacked on each other is in contact with another adjacent one of the brush pieces on side surfaces of the annular sections of the bush pieces adjacent to each other.

6. The nasal cavity cleaner according to claim 5, wherein each of the plurality of brush pieces further comprises a convex section is formed on the side surface of the annular section.

7. The nasal cleaner according to claim 1, wherein the brush holding section further comprises:
a wall section on the grip section side, the wall section on which the annular section of the brush abuts when the brush holding section is first inserted through the annular section; and
a pressurizing lid section pressurizing and holding the annular section of the brush piece along the central axis line toward the grip section when the brush holding section is last inserted through the annular section.

8. The nasal cavity cleaner according to claim 7, wherein the pressurizing lid section is formed by ultrasonic welding.

9. The nasal cavity cleaner according to claim 1, wherein the plurality of brush pieces are formed so that at least at an end opposite to the grip section, the brush bristles are shorter in length from the grip section side toward the side opposite to the grip section.

10. An oral cavity cleaner to be inserted into an oral cavity for cleaning the oral cavity, the oral cavity cleaner comprising:
a grip section to be gripped by a user;
a shaft-shaped brush holding section extending from the grip section; and
a plurality of brush pieces stacked on each other and held by the brush holding section,
wherein each of the plurality of brush pieces comprises:
an annular section formed in an annular shape; and
a plurality of brush bristles provided on an outer peripheral surface of the annular section, each of the plurality of brush pieces held by the brush holding section inserted through the annular section,
wherein the brush holding section comprises:
a base section in a cylindrical shape; and
a locking section extending on an outer peripheral surface of the base section along a central axis line of the brush holding section, the locking section formed in a rib shape having an outer diameter from the central axis line of the brush holding section to a top of the locking section, the outer diameter larger than an inner diameter of the annular section, and the locking section locked to an inner peripheral surface of the annular section of the brush piece held by the brush holding section to suppress a rotation of the brush piece around the central axis line,
wherein the brush holding section is inserted through the annular section by press fitting to lock the locking section to the inner peripheral surface of the annular section, and
wherein the locking section is crushed by the inner peripheral surface of the annular section with the brush holding section being inserted through the annular section.

11. The oral cavity cleaner according to claim 10, wherein the plurality of brush bristles are provided radially outward from the outer peripheral surface of the annular section over an entire circumference of the outer peripheral surface of the annular section.

12. The oral cavity cleaner according to claim 10, wherein on a cross-section perpendicular to the central axis line of the brush holding section, the top of the locking section is positioned between two interfaces between the locking section and the base section when viewed in at least one direction perpendicular to the central axis line.

13. The oral cavity cleaner according to claim 10, wherein the inner peripheral surface of the annular section of each of the plurality of brush pieces is pressed and expanded by the locking section with the brush holding section being inserted through the annular section.

14. The oral cavity cleaner according to claim 10, wherein each of the plurality of brush pieces stacked on each other is in contact with another adjacent one of the brush pieces on side surfaces of the annular sections of the bush pieces adjacent to each other.

15. The oral cavity cleaner according to claim 14, wherein each of the plurality of brush pieces further comprises a convex section is formed on the side surface of the annular section.

16. The oral cavity cleaner according to claim 10, wherein the brush holding section further comprises:
a wall section on the grip section side, the wall section on which the annular section of the brush piece abuts when the brush holding section is first inserted through the annular section; and
a pressurizing lid section pressurizing and holding the annular section of the brush piece along the central axis line toward the grip section when the brush holding section is last inserted through the annular section.

17. The oral cavity cleaner according to claim 16, wherein the pressurizing lid section is formed by ultrasonic welding.

18. The oral cavity cleaner according to claim 10, wherein the plurality of brush pieces are formed so that at least at an end opposite to the grip section, the brush bristles are shorter in length from the grip section side toward the side opposite to the grip section.
